(19) United States
(12) Patent Application Publication
(10) Pub. No.: US 2023/0059022 A1
(43) Pub. Date: Feb. 23, 2023

(54) PRUSSIAN BLUE ZnO CARBON NANOTUBE COMPOSITE FOR MEASURING HYDROGEN PEROXIDE IN CANCER CELLS

(71) Applicant: Middle Tennessee State University, Murfreesboro, TN (US)

(72) Inventors: Charles CHUSUEI, Murfreesboro, TN (US); Raja Ram PANDEY, Murfreesboro, TN (US)

(21) Appl. No.: 17/792,089

(22) PCT Filed: Jan. 6, 2021
(86) PCT No.: PCT/US21/12337

§ 371 Date: Jul. 11, 2022

Related U.S. Application Data
(60) Provisional application No. 62/959,517, filed on Jan. 10, 2020.

(51) Int. Cl. G01N 27/327; G01N 33/574; G01N 27/30; G01N 27/416; G01N 27/48

(52) U.S. Cl. CPC G01N 27/3278; G01N 33/57484; G01N 27/308; G01N 27/4161; G01N 27/48

(57) ABSTRACT

A Prussian blue/zinc oxide-carbon nanotube composite is provided, the nanotube composite being selective and sensitive for detection of hydrogen peroxide, which is important for screening for early cancer detection, monitoring cardiovascular disease, detecting onset of food spoilage, and enzymatic reactions that produce hydrogen peroxide as a byproduct. Also provided are methods using said zinc oxide-carbon nanotube composite in which standard addition is used in combination with chronoamperometry detection to quantify the level of hydrogen peroxide in a biological sample.

### CROSS-REFERENCE TO RELATED APPLICATION

This is an International Application under the Patent Cooperation Treaty, claiming priority to U.S. Provisional Patent Application No. 62/959,517, filed Jan. 10, 2020, the contents of which are incorporated herein by reference in their entirety.

### BACKGROUND

### 1. Field

The present disclosure relates to a zinc oxide-carbon nanotube composite that is both selective and sensitive for the detection of hydrogen peroxide, which is important for screening for oxidative stress, monitoring cardiovascular disease, detecting onset of food spoilage and enzymatic reactions that produce hydrogen peroxide as a byproduct.

### 2. Description of Related Art

Selective and quantitative measurements of hydrogen peroxide, an important reactive oxygen species (ROS), are involved in a host of biological redox reactions (Mikalai, M.; You, Z.; Vsevolod, V. B.; Dolph, L. H.; Vadim, N. G. PLoS ONE 2011, 6, 14564). A growing body of evidence suggests that oxidative stress, generating ROS (of which hydrogen peroxide is the most stable as compared to peroxides, superoxides, hydroxyl radicals and singlet oxygen), plays a key role regulating pathways in tumor cell survival (Reuter, S.; Gupta, S. C.; Chaturvedi, M. M.; Aggarwal, B. B. Free Rothe. Biol. Med. 2010, 49, 1603-1616). The ability to accurately measure hydrogen peroxide is important for understanding mechanisms underlying this phenomenon and thereby improve practical chemotherapy. However, matrix effects from interfering species in biological samples coupled with the transient nature of ROS hamper accurate measurements. Furthermore, standard immunoassays, which typically incorporate the use of fluorescent dyes, contribute to the complexity of the analyte solution. Despite recent improvements in fluorescent probes (Lippert, A. R.; Van de Bettner, G. C.; Chang, C. J. Acc. Chem. Res. 2011, 44, 793-804; Chen, X.; Tian, X.; Shin, I.; Yoon, J. Chem. Soc. Rev. 2011, 40, 4783-4804; and Chan, J.; Dodani, S. C.; Chang, C. J. Nat. Chem. 2012, 4, 973-984) and genetically encoded (Belousov, V. V.; Fradkov, F.; Lukyanov, K. A.; Staroverov, D. B.; Shakhbazov, K. S.; Terskikh, A. V.; Kukyanov, S. Nat. Methods 2006, 3, 281-286), very few ROS quantification studies are conducted in cancer cell media due to the lack of suitably accurate measurement techniques (Grisham, M. B. Comp. Biochem. Physiol., Part A: Mol. Integr. Physiol. 2013, 165, 429-438; Winterbourn, C. C. Biochim. Biophys. Acta 2014, 1840, 730-738

The solution to this technical problem is provided by embodiments characterized in the claims.

### SUMMARY

The present application relates to methods and compositions for measuring the level of reactive oxygen species, particularly hydrogen peroxide (H₂O₂), in biological samples, particularly cancer cells. The compositions comprise a nanocomposite comprising a Prussian blue (PB)/zinc oxide (ZnO) nanostructure attached to a carboxylic acid-functionalized multiwalled carbon nanotube (COOH-MWNT) for use in quantitating the amount of hydrogen peroxide in a biological sample. The methods of the invention further comprise use of the method of standard addition in combination with chronoamperometry detection to quantify the level of hydrogen peroxide in a biological sample using the PB/ZnO/COOH-MWNT nanocomposite.

Embodiments of the present invention comprise:
1. A composition comprising an electrode having attached thereto a nanocomposite comprising a hydrogen peroxide catalyst and a zinc oxide nanostructure attached to a carboxylic acid-functionalized multiwalled carbon nanotube (ZnO/COOH-MWNT).
2. The composition of embodiment 1, wherein the electrode is a glass-like carbon electrode.
3. The composition of embodiment 1 or 2, wherein the hydrogen peroxide catalyst is Prussian blue.
4. The composition of any of embodiments 1-3, wherein the zinc oxide nanostructure has an average diameter of about 50 nm to about 60 nm.
5. The composition of any of embodiments 1-4, wherein the carboxylic acid-functionalized multiwalled carbon nanotube has a diameter of about 30 nm.
6. The composition of embodiment 3, wherein the ratio of Prussian blue to ZnO/COOH-MWNT is about 1:2.
7. A method of preparing a nanocomposite, comprising:
   a) preparing a zinc oxide nanostructure;
   b) attaching the zinc oxide nanostructure to a carboxylic acid-functionalized multiwalled carbon nanotube; and
   c) attaching a hydrogen peroxide catalyst to the carboxylic acid-functionalized multiwalled carbon nanotube.

The method of embodiment 7, wherein step (b) is performed by ultrasonication for approximately 60 minutes.
9. The method of embodiment 7 or 8, wherein step (c) is performed at a pH of 6.6.
10. The method of embodiment 9, wherein step (c) is performed over a period of about 5 hours.
11. The method of any of embodiments 7-10, wherein the hydrogen peroxide catalyst is attached to the nanotube electrostatically.
12. The method of any of embodiments 7-11, wherein the hydrogen peroxide catalyst is Prussian blue.
13. The method of any of embodiments 7-12 further comprising depositing the nanocomposite onto an electrode.
14. The method of embodiment 13, wherein the electrode is a glass-like carbon electrode.
15. A method for quantitating the level of hydrogen peroxide in a biological sample, the method comprising:
   a) generating a standard curve for hydrogen peroxide concentration by
      i. adding serial concentrations of hydrogen peroxide to a buffer solution,
      ii. inserting the electrode of any of embodiments 1-6 into the solution,
      iii. measuring the concentration of hydrogen peroxide using an electrochemical sensor, and
      iv. plotting the resulting current at each concentration of hydrogen peroxide to generate the standard curve; and
   b) determining the concentration of hydrogen peroxide in the biological sample by
      i. inserting the electrode of any of embodiments 1-6 into the biological sample,
      ii. detecting hydrogen peroxide through the electrode using an electrochemical sensor, and
      iii. determining the concentration of hydrogen peroxide by comparing the results of step (b)(ii) to the standard curve.
16. The method of embodiment 15, wherein the electrochemical sensor is a cyclic voltammeter or a chronoamperometer,
17. The method of embodiment 15 or 16, wherein hydrogen peroxide is detected in the biological sample at a concentration of at least 1 μM.
18. The method of any of embodiments 15-17, wherein the biological sample is from a subject having or suspected of having cancer, or is from an immortalized cancer cell line.
19. The method of embodiment 18, wherein the cancer is breast cancer.
20. The method of any of embodiments 15-19, wherein the hydrogen peroxide is detected in the biological sample in the range of about 1 μM to about 21 μM.
21. The method of any of embodiments 15-20, wherein the hydrogen peroxide is quantitated in the biological sample within about 15 minutes.
22. The method of any of embodiments 15-21, wherein no matrix effect is observed.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements.

FIGS. 1A and 1B show cyclic voltammetry (CV) measurements in 5 mM hydrogen peroxide at 50 mV·s−1 using ZnO/COOH-MWNTs: (FIG. 1A) effect of sonication time; (FIG. 1B) point of zero charge (PZC) of Prussian Blue (PB) and a 60 min sonicated ZnO/COOH-MWNT composite for electrostatic attachment.

FIGS. 2A-2C show CVs of 5 mM hydrogen peroxide at pH 7.0 showing (FIG. 2A) the effect of PB to ZnO/COOH-MWNTs ratios (by mass), (FIG. 2B) stirring time for PB to attach to ZnO/COOH-MWNTs, and (FIG. 2C) a plot indicating that a 5-h stirring time was needed for attaching PB to ZnO/COOH-MWNTs for optimum sensitivity.

FIGS. 3A-3C show TEM images of: (FIG. 3A) ZnO; (FIG. 3B) ZnO/COOH-MWNTs; and, (FIG. 3C) histogram showing the average diameter of refluxed ZnO attached to the COOH-MWNTs. Using ImageJ software ver. 1.46r (National Institutes of Health: Bethesda, Md., USA), the diameter of ZnO was found to be 12.7±0.1 nm as shown in the histogram (FIG. 3C) of the nodules tethered to the COOH-MWNTs (FIG. 3B), which were confirmed by EDX to consist of ZnO.

FIGS. 4A-4C show an X-ray photoelectron spectroscopy (XPS analysis) of (FIG. 3A) O 1s, (FIG. 3B) Zn 2p, and (FIG. 3C) Fe 2p core level binding energies of ZnO, ZnO/COOH-MWNTs, and PB/ZnO/COOH-MWNTs.

FIGS. 5A and 5B represent an analysis of hydrogen peroxide under CV at pH 7.0 with a 50 mV·s−1 scan rate (FIG. 5A) using (a) PB/ZnO/COOH-MWNTs with 5 mM hydrogen peroxide in phosphate buffer solution (PBS), (b) ZnO/COOH-MWNTs with 5 mM hydrogen peroxide in PBS, (c) PB/ZnO/COOH-MWNTs in PBS only, (d) PB with 5 mM hydrogen peroxide in PBS, (e) glassy carbon electrode (GCE) with 5 mM hydrogen peroxide in PBS, and (FIG. 5B) a plot of pH vs current to show optimum pH response for the highest electrocatalytic activity at pH 7.0 using CVs at reduction and oxidation potentials of −0.004 V and +0.277 V, respectively.

FIGS. 6A-6C represent an analysis of hydrogen peroxide with chronoamperometric sensing (CA) at pH 7.0 using PB/ZnO/COOH-MWNTs: (FIG. 6A) CA plot showing the detection of hydrogen peroxide from 1 μM to 3 mM; (FIG. 6B) CA calibration curve hydrogen peroxide (red circle denotes deviation from linearity); (FIG. 6C) standard addition validation control plot at pH 7.0 using PB/ZnO/COOH-MWNTs.

FIGS. 7A-7D show a comparison of concentrations of hydrogen peroxide in Dox-treated and untreated cancer cells. The bar graphs summarize measurements of hydrogen peroxide release from (FIG. 7A) BT20 cells with CA self-assembled monolayer standard addition method (SAM), (FIG. 7B) BT20 cells with ELISA, (FIG. 7C) 4T1 cells with CA SAM, and (FIG. 7D) 4T1 cells with ELISA.

FIGS. 8A and 8B represent a control CA study of hydrogen peroxide decomposition in PBS, BT20, and 4T1 cancer cells upon addition of 3 mM hydrogen peroxide (FIG. 8A). Real time CA measurements of hydrogen peroxide were made using the PB/ZnO/COOH-MWNT sensor, and (FIG. 8B) CA selectivity study of hydrogen peroxide using PB/ZnO/COOH-MWNTs at pH 7.0. Interferents include uric acid (UA), ascorbic acid (AA), acetaminophen (APAP), folic acid (FA), and glucose (Glu).

### DETAILED DESCRIPTION

Before the subject disclosure is further described, it is to be understood that the disclosure is not limited to the particular embodiments of the disclosure described below, as variations of the particular embodiments may be made and still fall within the scope of the appended claims. It is also to be understood that the terminology employed is for the purpose of describing particular embodiments and is not intended to be limiting. Instead, the scope of the present disclosure will be established by the appended claims.

In this specification and the appended claims, the singular forms “a,” “an,” and “the” include plural reference unless the context clearly dictates otherwise. Unless defined otherwise, all technical and scientific terms used herein have the same meaning as commonly understood to one of ordinary skill in the art to which this disclosure belongs.

Provided herein are methods and compositions for detection of reactive oxygen species in a biological sample. The compositions, and methods of use thereof, comprise a nanocomposite comprising a hydrogen peroxide catalyst and a zinc oxide nanostructure attached to a carboxylic acid-functionalized multiwalled carbon nanotube useful for rapid assaying of reactive oxygen species generated in biological samples. For the purposes of the present invention, a “biological sample” refers to a sample collected from a subject having or suspected of having cancer (such as, but not limited to, from biopsies, aspirates, blood, serum, or any other sample taken from a subject) and also includes immortalized cell lines collected from a cancer patient.

In specific embodiments, the methods and compositions are useful for measuring oxidative stress in a cell. Oxidative stress activates inflammatory pathways which can lead to transformation of a normal cell to a tumor cell, tumor cell survival, proliferation, chemoresistance, radioresistance, invasion, angiogenesis and stem cell survival. Thus, the present invention provides a mechanism for monitoring oxidative stress in, for example, a tumor environment to elucidate mechanisms of action of ROS on tumor cells, to monitor progression of a tumor cell, to monitor response to treatment of the tumor, and the like. The invention is further useful as a selective and sensitive method for monitoring cardiovascular disease, detecting onset of food spoilage, and for evaluating enzymatic reactions that produce ROS as a byproduct.

In some embodiments, the ROS is hydrogen peroxide, which has been associated with tumor cell survival. The combination of hydrogen peroxide's transient nature along with matrix effects makes monitoring this molecule in biological samples a challenge. The present invention addresses these obstacles, in part, by combining the standard addition method (SAM) with chronoamperometric sensing (CA) as described elsewhere herein.

In certain embodiments, the hydrogen peroxide is detected in the biological sample at a concentration of at least 1 μM, at least 2 μM, at least 3 μM, at least 4 μM, at least 5 μM, or from about 1 μM to about 21 μM, from about 1 μM to about 15 μM, or from about 1 μM to about 10 μM.

The compositions (and methods of use thereof) comprise a nanoparticle composite (“nanocomposite”) comprising a hydrogen peroxide catalyst and a zinc oxide nanostructure attached to a carboxylic acid-functionalized multiwalled carbon nanotube (ZnO/COOH-MWNT). Preparation of the ZnO/COOH-MWNT nanocomposite is described elsewhere herein and in US Patent Publication No. 20150129426, which is herein incorporated by reference in its entirety. US Patent Publication No. 20150129426 further describes and defines a zinc oxide nanostructure. In one aspect, the zinc oxide nanostructure has an average diameter of from about 20 nm to about 80 nm. In one aspect, the zinc oxide nanostructure has an average diameter of about 50 nm to about 60 nm. In one aspect, the carboxylic acid-functionalized multiwalled carbon nanotube has a diameter of about 30 nm.

In various embodiments of the invention, the ZnO/COOH-MWNT composite is deposited onto an electrode. The electrode can comprise any sufficiently conductive material, such as metals, semiconductors, graphite, conductive polymers, and the like. Preferably, the electrode surface will have high temperature resistance, hardness (for example, >7 Mohs), low density, low electrical resistance, low friction, and/or low thermal resistance. In one embodiment, the nanoparticle composite is deposited onto a glass-like carbon (also referred to as “glassy carbon” or “vitreous carbon”) electrode. In certain embodiments, the attachment of the ZnO/COOH-MWNT composite is performed using sonication or ultrasonication for a period of about 30, about 45, about 60, about 75, about 90, about 105, about 120, about 130, about 140, about 150, or more minutes. In specific embodiments, the sonication is performed for about 60 minutes.

In one aspect, the electrode has a peak current (Iₚ) of at least about 0.2 mA. In one aspect, the electrode has a peak current (Iₚ) of at least about 0.4 mA. In one aspect, the electrode has a peak current (Iₚ) of at least about 0.5 mA. In one aspect, the electrode has an electroactive surface area of at least about 0.9 cm². In one aspect, the electrode has an electroactive surface area of at least about 1.4 cm². In one aspect, the electrode has a reduction potential peak (E_{c}) of about −430 mV or greater versus Ag/AgCl (3.5 M KCl). In one aspect, the electrode has a reduction potential peak (E_{c}) of about −360 mV or greater versus Ag/AgCl (3.5 M KCl).

In specific embodiments, the nanocomposite further comprises a hydrogen peroxide catalyst. In one embodiment, the hydrogen peroxide catalyst is Prussian blue (PB). The addition of PB to the composite improves the reduction of hydrogen peroxide in the electrochemical sensing reaction by enhancing electron transfer to the ZnO/COOH-MWNT composite. In various embodiments, the PB is electrostatically attached to the COOH-MWNT surface. The ratio of PB to ZnO/COOH-MWNT can range from about 0.5:1, about 1:1, about 1.5:1, about 2:1, about 3:1, about 1:3, about 1:2, about 1:1.5, about 1:1, about 1:0.5. In one embodiment, the ratio of PB to ZnO/COOH-MWNT is 1:2. The PB can be attached in an induction reaction at a pH of about 6.3, 6.4, 6.5, 6.6, 6.7, 6.8, or 6.9. In one embodiment, the induction is performed at a pH of 6.6. The induction time can be about 2 hours, about 3 hours, about 4 hours, about 5 hours, about 6 hours, about 7 hours, about 8 hours, about or longer. In various embodiments, the induction time for attachment of PB is 5 hours.

In specific embodiments of the present invention, quantitation of the reactive oxygen species is performed using an electrochemical sensor. An “electrochemical sensor” is a device configured to detect the presence of and/or measure the concentration of an analyte via electrochemical oxidation and reduction reactions. These reactions are transduced to an electrical signal that can be correlated to an amount or concentration of analyte. In various embodiments, the quantitation is performed using chronoamperometry (CA) or cyclic voltammetry (CV), or variants thereof. CA is an electrochemical method in which the potential of the working electrode is stepped and the resulting current from faradaic processes occurring at the electrode (caused by the potential step) is monitored as a function of time. The functional relationship between current response and time is measured after applying single or double potential step to the working electrode of the electrochemical system (Bard, A. J.; Larry R. Faulkner (2000). Electrochemical Methods: Fundamentals and Applications (2 ed.). Wiley. CV is an electrochemical method which measures current that develops in an electrochemical cell under conditions where voltage is in excess of that predicted by the Nernst equation. CV is performed by cycling the potential of a working electrode and measuring the resulting current (Skoog, D.; Holler, F.; Crouch, S. Principles of Instrumental Analysis 2007). Methods for using CA and CV are known in the art and described elsewhere herein.

In specific embodiments of the present invention, the quantitation of reactive oxygen species in the biological samples is performed using CA in combination with the standard addition method (SAM). SAM is a type of quantitative analysis approach whereby the standard (e.g., hydrogen peroxide) is added directly to aliquots of the sample to be analyzed. A particular advantage of this method is that it reduces or avoids sample matrix effects. Sample matrix effects occur when sample components other than the target analyte contribute to the analytical signal, which makes it challenging to accurately compare the analytical signal between the biological sample and the standard using the traditional calibration curve approach.

In the present invention, samples are measured by standard additions of hydrogen peroxide in increasing order to create the calibration curve needed to determine the unknown hydrogen peroxide concentration in the biological samples. A calibration curve (i.e., standard curve) can be generated using serial dilutions of known concentrations of the target analyte (e.g., hydrogen peroxide). In some embodiments, the calibration curve is generated using at least about 4, at least about 5, at least about 6, at least about 7, about 8, about 9, about 10, or more known concentrations of the target analyte.

SAM is typically applied to atomic absorption, fluorescence spectroscopy, ICP-OES and gas chromatography. There are few literature reports in which SAM is applied to CA. To date, only one group has successfully coupled SAM with CA for measuring hydrogen peroxide. Li et al. used a PB carbon nanotube composite to do so (Zbiljic, J.; Guzsvány, V.; Vajdle, O.; Prlina, B.; Agbaba, J.; Dalmacija, B.; Kónya, Z.; Kalcher, K. J. Electroanal. Chem. 2015, 755, 77-86). The analysis range achieved in this study, however, has a lower limit of 10 μM, which is still insufficiently sensitive for analyzing oxidative stress in cancer cell lines. Furthermore, the electrochemical technique relies heavily on the Fenton reaction for hydrogen peroxide quantitation. In applications involving ROS probes in cancer cell media, this feature would hamper ROS analysis due to generation of additional ROS by the PB-based electrocomposite. To address this deficiency, the present invention incorporates ZnO upon which hydrogen peroxide redox will largely take place.

The following examples are offered by way of illustration and not by way of limitation.

### Experimental Examples

### Cell Culture

BT20 and 4T1 cells were purchased from American Type Culture Collection (ATCC). Both cell lines were maintained in RPMI-1640 medium (Sigma-Aldrich) with 10% Fetal Bovine Serum (FBS) (Gibco) at 37° C. with 5% CO₂. The medium was renewed every two days. Well-grown BT20 cells in logarithmic phase were digested by 0.25% (w/v) Trypsin (Fisher Scientific) from the original culture flask and mixed well before being seeded on a 96-well cell culture plate (Denville Scientific) with a density of 5×10⁴ cells/mL and incubated with the same full medium at 37° C. with 5% CO₂ for 24 h. Cells were then treated with 100 μL of 50 μM doxonibicin (Dox) (Sigma-Aldrich). Cells treated with 100 μL of vehicle (RPMI-1640 medium) only served as a control. The Dox group and control group each had eight replicate wells. After 24 h of incubation, 50 μL of the medium was collected from each well and proceeded for hydrogen peroxide determination.

### Hydrogen Peroxide Release Assay

The hydrogen peroxide release assay was carried on using Amplex™ Red Hydrogen Peroxide/Peroxidase Assay Kit (Invitrogen) according to the instruction. The hydrogen peroxide standards for standard curve were prepared by diluting the 20 mM hydrogen peroxide stock solution with 1× reaction buffer. The final concentrations of seven hydrogen peroxide standards were 10, 5, 2.5, 1.25, 0.625, 0.3125, and 0 μM, respectively. After 50 μL of sample medium and 50 μL of each standard were loaded into 96-well plate, 50 μL of the working solution (0.1 mM Amplex™ Red reagent, 0.02 U/mL. Horseradish peroxidase, and 1× reaction buffer) were added into these wells. After incubation at room temperature in the dark for 30 min, the absorbance of each well at 560 nm of was measured with a CLARIOstar™ microplate reader (BMG Labtech). The concentration of hydrogen peroxide in the sample was calculated according to the standard curve. T-test was applied for the analysis of the statistical difference between the Dox group and control group.

### Electrode Preparation

The nanocomposites were deposited onto glassy carbon electrode (GCE) surfaces for electrochemical analysis of hydrogen peroxide. Synthesizing the composite was achieved in three steps: (i) ZnO nanoparticles were prepared by refluxing, which were then (ii) attached to COOH-MWNTs, and followed by (iii) PB electrostatic attachment to the COOH-MWNTs. The synthesis of refluxed ZnO was performed using a procedure developed by Das et al. (Chemosensors 2018, 6, 65-77). A 42-mL volume of 1 M NaOH was added to a round bottle flask. A separatory funnel with 0.5 M of 42-mL of Zn(NO₃)₂.6H₂O was connected to the flask. To obtain the ZnO NPs, Zn(NO₃)₂.6H₂O was passed dropwise for 60 min under constant stirring (with a magnetic stirrer) under inert N₂ atmosphere while NaOH solution was heated to 100° C. This refluxing process was continued under these conditions for an additional 2 h at 100° C. After getting white precipitate, the ZnO was filtered and washed with Millipore water. After a number of washings, prepared NPs were dried in a desiccator overnight. ZnO NPs were again dried in an oven for another hour at 65° C.

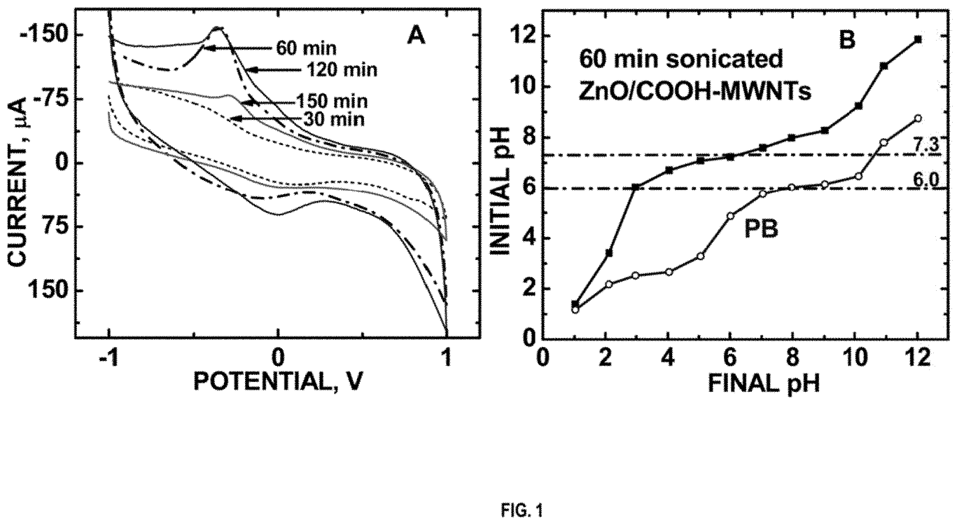

Equal masses of refluxed ZnO and COOH-MWNTs (4.0 mg of each) were vortex mixed in 1.0 mL. AAEA solvent using a polyethylene tube to prepare the ZnO/COOH-MWNT electrocatalyst composite. Sonication was employed to tether the ZnO nanoparticles to the COOH-MWNT surface. To optimize and identify the ideal ZnO/COOH-MWNT composite for hydrogen peroxide sensing, GCEs were prepared by depositing 30, 60, 120 and 150-min separately sonicated composites. It was discovered that the optimum sonication time was 60 min (FIG. 1A). The composite was completely dried at 80° C. for 3 h in an oven, followed by additional drying in the desiccator for 24 h.

After drying the composite, 4.0 mg of ZnO/COOH-MWNTs and 2.0 mg of PB were added to the 1.0 mL of PBS at pH 6.6 for attachment (FIG. 1B) and stirred for 5 h using a magnetic stirrer in small glass vials. The PZC of the ZnO/COOH-MWNTs and PB was determined applying a procedure developed by Park and Regalbuto (J. Colloid Interface Sci. 1995, 175, 239-252; McPhail, M. R.; Sells, J. A.; He, Z.; Chusuei, C. C. J. Phys. Chem. C 2009, 113, 14102-14109; Deb, A. K.; Das, S. C.; Saha, A.; Wayu, M. B.; Marksberry, M. H.; Baltz, R. J.; Chusuei, C. C. J. Appl. Electrochem. 2016, 46, 289-298). The procedure is summarized as follows. With the help of dilute aqueous solutions of HCl and NaOH, 12 solutions in the range of 1.0 to 12.0 pH were prepared. Polyethylene vials were filled with 1.8 mL aliquots of each solution and equilibrated for 1 h. The initial pH of each solution was then measured. To each vial, 2.0 mg of PB or ZnO/COOH-MWNTs to be analyzed was added. The vials were capped and mixed with a Vortex mixer, and left for an additional 16 h equilibration period. Using a spear-tip semisolid electrode, the final pH of PB or ZnO/COOH-MWNTs was recorded for each vial. Plateaus obtained from the plot of initial vs final pH denoted the PZC.

### Material Characterization

Transmission electron microscopy (TEM) analysis was performed using a Hitachi H-7650 TEM operated at 100 kV with 80,000× magnification. TEM image of the synthesized ZnO and ZnO/COOH-MWNTs are studied. ZnO was deposited on and anchored to the outside of MWNTs. These images of ZnO nanoparticles with the average diameter are calculated in the histogram diagram using ImageJ software (ver. 1.46r, Java 1.6.0; National Institutes of Health, Bethesda, Md., USA). XPS was used to characterize the PB/ZnO/COOH-MWNT composite. X-ray photoelectron spectroscopy (XPS) were acquired using a Perkin-Elmer PHI 560 system with a double-pass cylindrical mirror analyzer. X-rays were generated using a Mg Kα anode with a hv=1253.6 eV photon energy, operated at 250 W and 13 kV. The C 1s core level at 284.4 eV denoting the sp² C—C bonding within the graphene sheets of the MWNTs was used as a charge reference. Shirley background subtractions for the C 1s and O 1s core levels were used. Tougaard background subtractions for the Zn 2p and Fe 2p core levels were applied. Deconvolutions were performed using 70%-to-30% Gaussian-Lorentzian line-shapes using CasaXPS software, version 2.2.107 (Devon, United Kingdom). The ultrahigh vacuum system pressure did not exceed 1×10⁸ Torr during XPS scans. High resolution narrow scans for C 1s, O 1s, Zn 2p and Fe 2p were carried out. Atomic percent composition measured from the C 1s, O 1s, Zn 2p and Fe 2p orbitals, after normalizing their integrated peak areas to their atomic sensitivity factors were 17.2%, 82.5%, 0.25% and 0.04%, respectively.

Electrochemical activity of the PB/ZnO/COOH-MWNTs was studied using cyclic voltammetry (CV) and chronoamperometry (CA) using AfterMath™ software ver 1.2.5658 and a WaveNano™ potentiostat (Pine Research Instrument Co., Raleigh, N.C., USA). A custom-built Faraday cage constructed of Cu grid mesh was used to reduce external electromagnetic interference. The three-electrode electrochemical cell consisted of a Ag/AgCl (3.5 M KCl) reference electrode, a counter electrode made of platinum wire, and a PB/ZnO/COOH-MWNTs/GCE working electrode stored in inert N₂ atmosphere until usage. CVs of the cell were studied in the range of potentials from −1.0 V to +1.0 V using a 50 mV·s⁻¹ scan rate. The optimum peak potential based on CV result for hydrogen peroxide detection was used for CA analysis. Hydrogen peroxide concentrations of 1 μM to 3 mM were used since these concentrations are for studying oxidative stress of hydrogen peroxide in cancer cell line purposes. The PB/ZnO/COOH-MWNT composite was used as the working electrode for chronoamperometric measurements. The concentrations of hydrogen peroxide in BT20 cancer cells were measured chronoamperometrically, employing the standard addition method (CA SAM). Samples were measured by standard additions of hydrogen peroxide in increasing order to create the calibration curve needed to determine the unknown hydrogen peroxide concentration in the cancer cells. BT20 and 4T1 cancer cells were cultured and the concentration of hydrogen peroxide in the cancer cells analyzed using this sensor. Potentials for optimized CA measurements were obtained from CV data at the maximum signal.

### Results and Discussion

In the CV of the 5 mM hydrogen peroxide solution in PBS (FIG. 1A), the 60 min sonicated composite showed optimum sensitivity (based on measured CV relative peak-to-peak heights) to hydrogen peroxide due to electrocatalytic reactions with ZnO nanoparticles on the surface of MWNTs as shown in FIG. 1A. The signal intensity of the 5 mM concentration of hydrogen peroxide varied as a function of sonication time. Signal intensity as measured by the peak-to-peak height increased from 30 to 60 min, and then decreased from 60 to 150 min of sonication. Maximum signal intensity for the hydrogen peroxide was achieved at 60 min of sonication. Trial and error experiments showed an induction period of 5 h and a 1-to-2 PB-to-ZnO/COOH-MWNT ratio produced the optimized composite formed (vide infra). After the optimized ZnO/COOH-MWNTs were produced, PB was electrostatically attached to the COOH-MWNT surface, based on differences in the isoelectric points of the two materials. The PZC of the ZnO/COOH-MWNT composite was 7.3 whereas that of PB was 6.0 (FIG. 1B). An intermediate pH value of 6.6 was applied to attach PB to the composite. Under these conditions the PB adopts a negative surface charge while the ZnO/COOH-MWNTs adopts a positive surface charge to serve as driving forces for Coulombic attachment. A PBS solution adjusted to pH=6.6 was used to combine the PB to the ZnO/COOH-MWNTs. Following Gouy-Chapman theory, at this pH value the PB adopts a negative surface charge while the ZnO/COOH-MWNTs adopt a positive surface charge to serve as driving forces for composite formation. The attachment of PB resulted in >2-fold increase in hydrogen peroxide signal.

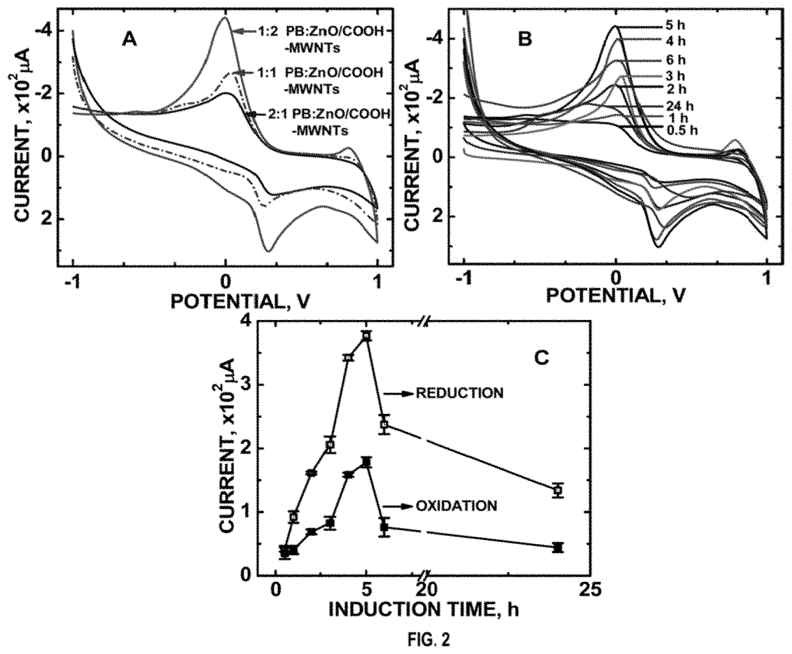

FIG. 2A shows differences in CV signal as a function of various PB loading onto the ZnO/COOH-MWNTs nanocomposite with measurements across the working electrode versus Ag/AgCl reference electrode in phosphate buffer solution (PBS) at pH 7.0. Highest electrocatalytic activity was observed at (1:2) mass ratio of PB:ZnO/COOH-MWNTs composite (FIG. 2A). FIG. 2B shows a series of CVs at room temperature for different induction periods (combining PB with ZnO/COOH-MWNTs while stirring in a reactor) to form the PB/ZnO/COOH-MWNT composite. Maximum current was achieved with a 60 min sonication time to tether the ZnO to the COOH-MWNTs (FIG. 1A). A greater than two-fold signal enhancement for hydrogen peroxide reduction by the sensor was achieved by using a 5-h induction time to attach PB to the ZnO/COOH-MWNTs (FIGS. 2B and 2C).

### Surface Characterization of ZnO NPs, ZnO/COOH-MWNTs and PB/ZnO-COOH-MWNTs

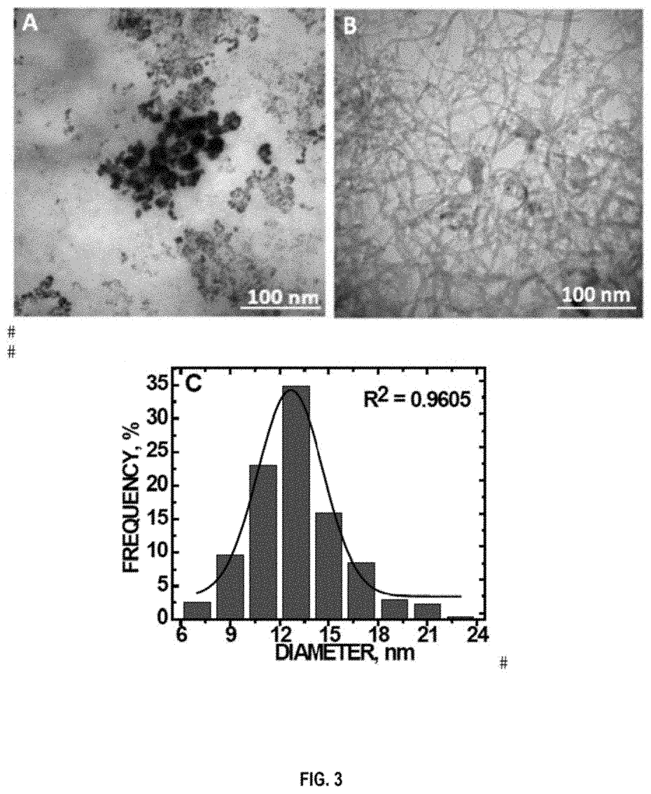

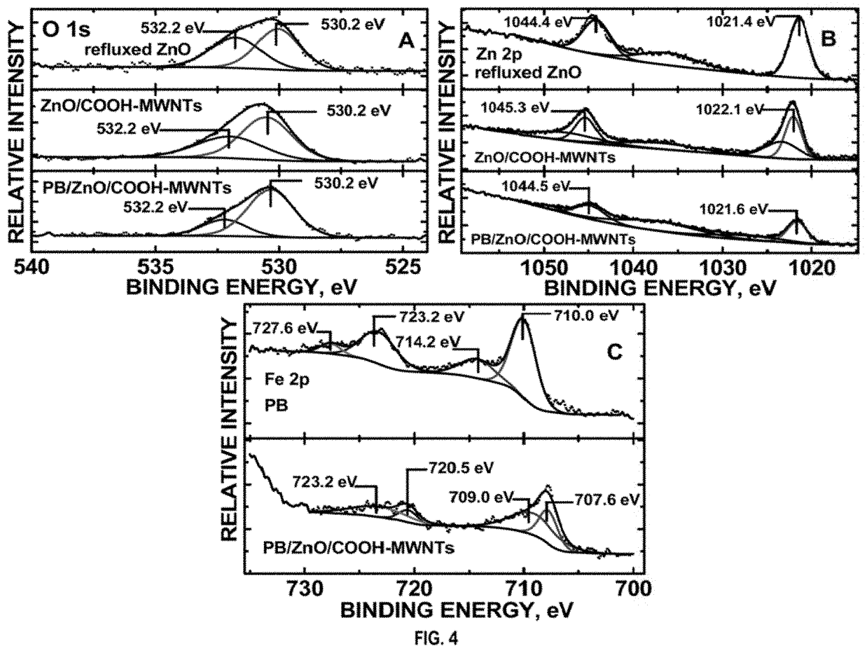

Morphological structures of refluxed ZnO and ZnO/COOH-MWNTs were investigated using TEM as shown in FIGS. 3A and 3B, respectively. The chemical composition and information regarding oxidation states of atoms in the PB/ZnO/COOH-MWNTs nanocomposite were acquired using XPS. FIG. 4A shows O 1s core levels at 530.2 eV, denoting ZnO and 532.2 eV emanating from adsorbed hydroxyl O atoms from exposure to aqueous solution during refluxed ZnO NP synthesis. There is an observable decrease in integrated peak area intensity in the 532.2 eV chemical oxidation state upon attachment of the PB to the ZnO/COOH-MWNT composite surface. The 532.2 eV binding energy (BE) relative peak area decreased from 39.9% in the O 1s spectrum for ZnO/COOH-MWNTs to 24.5% for the corresponding peak envelope in the O 1s spectrum for PB/ZnO/COOH-MWNTs. While not being bound to any particular theory or mechanism, it is postulated that this decrease in relative peak area is a result of attenuation by PB as the PB molecule interacted with adsorbed hydroxyls on the ZnO/COOH-MWNTs.

The Zn 2p doublet separation value (23.0 eV) is indicative of refluxed ZnO (FIG. 4B). The peak positions of Zn 2p from the ZnO/COOH-MWNT nanocomposite shift to higher BE as compared to those of ZnO, denoting the withdrawal of electron density from ZnO by the COOH groups on the MWNT surface that coordinate with the ZnO in the formation of composite. Furthermore, the observed ˜0.8 eV chemical shift towards lower BE with the attachment of PB of ZnO/COOH-MWNT denotes an increase electron density in the Zn 2p_{1:2} orbitals from 1045.3 eV to 1044.5 eV, and in the Zn 2p_{3:2} orbitals from 1022.1 to 1021. 6 eV (FIG. 4B), which is consistent with ZnO reduction by PB. In examining the Fe 2p_{1:2} and Fe 2p_{3:2} core levels, large (˜3.0 eV) BE shifts are observed from 723.2 to 720.5 eV and 710.0 to 707.6 eV (FIG. 4C), respectively, in comparing PB with the PB/ZnO/COOH-MWNT composite, indicative of chemical bonding upon PB attachment to the ZnO/COOH-MWNT surface. The XPS data showed that atomic percent composition of O 1s, C 1s, Zn 2p, and Fe 2p was 82.5%, 17.2%, 0.25%, and 0.04%, respectively (Table 1). Electron transfer from Fe(III) to Fe(II), contributing to the formation of the reduced PB state, which in turn is responsible for the reduction of hydrogen peroxide in the electrochemical sensing reactions:

$${{Fe}_{4}^{lll} [ {{Fe}^{ll} ( CN )}_{6} ]}_{3} + 4 {e}^{-} + 4 {K}^{+} {\to }^{ZnO / COOH - MWNTs} {K}_{4} {{Fe}_{4}^{ll} [ {{Fe}^{ll} ( CN )}_{6} ]}_{3} \qquad ( 1 )$$

$${{Fe}_{4}^{lll} [ {{Fe}^{ll} ( CN )}_{6} ]}_{3} + 2 {H}_{2} {O}_{2} ⇄ {K}_{4} {{Fe}_{4}^{lll} [ {{Fe}^{ll} ( CN )}_{6} ]}_{3} + 4 {OH}^{-} + 4 {K}^{-} \qquad ( 2 )$$

In other words, the addition of PB to the composite improves the reduction of hydrogen peroxide in the electrochemical sensing reaction by enhancing electron transfer to the ZnO/COOH-MWNT composite, which is corroborated by the electrochemical results (vide infra).

TABLE 1
XPS core level shift spectral summary Prussian Blue (PB)

| | | BE peaks (fwhm, % |
|---|---|---|
| orbitals | atom % | integrated peak area) |
| C 1s | 67.7 | 284.7 eV (2.6, 84.6%), |
| | | 287.5 eV (3.1, 15.4%) |
| O 1s | 1.6 | 532.4 eV (3.2, 100%) |
| N 1s | 29.0 | 397.6 eV (2.4, 65.6%), |
| | | 402.3 eV (3.9, 34.4%) |
| Fe 2p | 1.54 | 710.0 eV (2.5, 48.5%), |
| | | 723.2 eV (3.7, 28.9%), |
| | | 714.2 eV (3.7, 17.2%), |
| | | 727.6 eV (2.2, 5.37%) |

Refluxed ZnO

| | | |
|---|---|---|
| C 1s | 22.8 | 284.7 eV (2.7, 100%) |
| O 1s | 3.8 | 530.2 eV (2.0, 53.3%), |
| | | 532.2 eV (2.4, 46.7%) |
| Zn 2p | 73.3 | 1021.4 eV (2.6, 57.4%), |
| | | 1044.4 eV (3.5, 42.6%) |

ZnO/COOH-MWNTs

| | | |
|---|---|---|
| C 1s | 91.7 | 284.4 eV (2.0, 73.4%), |
| | | 286.5 (2.2, 10.7%), |
| | | 289.0 eV (4.1, 15.9%) |
| O 1s | 7.16 | 530.2 eV (2.3, 60.1%), |
| | | 532.2 eV (3.1, 39.9%) |
| Zn 2p | 1.10 | 1022.1 eV (2.1, 57.9%), |
| | | 1045.3 eV (2.6, 42.1%) |

PB/ZnO/COOH-MWNTs

| | | |
|---|---|---|
| C 1s | 17.2 | 284.4 eV (2.0, 37.3%), |
| | | 285.2 eV (2.4, 62.7%) |
| O 1s | 82.5 | 532.2 eV (2.0, 24.5%), |
| | | 530.2 eV (2.2, 75.5%) |
| Zn 2p | 0.25 | 1021.6 eV (2.6, 53.1%), |
| | | 1044.5 eV (3.5, 46.9%) |
| Fe 2p | 0.038 | 707.6 eV (2.0, 25.7%), |
| | | 720.5 eV (2.0, 9.4%) |
| | | 709.0 eV (4.9, 41.1%), |
| | | 723.2 eV (5.2, 23.7%) |

### Electrocatalytic Characteristics and Optimization of the Sensor.

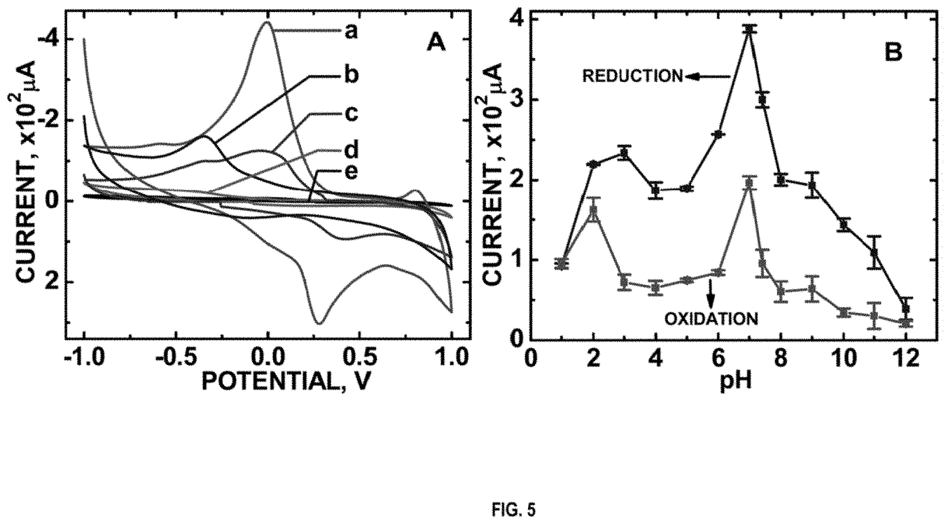

The electrochemical response of PB/ZnO/COOH-MWNT/GCE surface with hydrogen peroxide was compared with control experiments. As shown in FIG. 5A, point e, there was no electrochemical behavior to hydrogen peroxide on the bare GCE at given potential in PBS (pH 7.0) solution. The cathodic and anodic current peaks were observed at −0.004V and +0.277 V vs Ag/AgCl, respectively, which had a pronounced electrochemical response when GCE was modified with PB/ZnO/COOH-MWNTs (FIG. 5A, point a). Electrochemically controlled experiments were performed using ZnO/COOH-MWNTs and PB in which the PB/ZnO/COOH-MWNTs composite had increased sensitivity (FIG. 5A).

Symmetric peak shapes in the CVs at various pH conditions denoted quasi-reversible redox processes. During CV, hydrogen peroxide is oxidized to hydroxide, which is then reduced back to hydrogen peroxide via a two-electron process (J. Colloid Interface Sci. 1995, 175, 239-252; McPhail, M. R.; Sells, J. A.; He, Z.; Chusuei, C. C. J. Phys. Chem. C 2009, 113, 14102-14109; Deb, A. K.; Das, S. C.; Saha, A.; Wayu, M. B.; Marksberry, M. H.; Baltz, R. J.; Chusuei, C. C. J. Appl. Electrochem. 2016, 46, 289-298), in which 2 moles of OH⁻ are generated from 1 mole of hydrogen peroxide. FIG. 5B shows the amperometric response of the PB/ZnO/COOH-MWNTs/GCE as a function of pH in 5 mM hydrogen peroxide at reduction and oxidation potentials of −0.004 V and +0.277 V vs Ag/AgCl, respectively. The cathodic and anodic currents are maximized at pH 7.0 for both oxidation and reduction potentials.

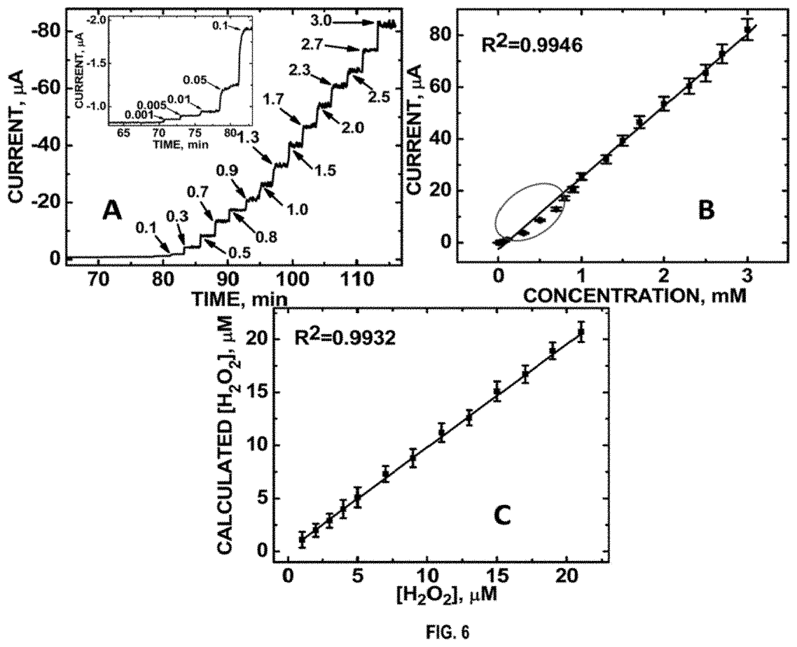

FIG. 6A shows a typical current vs time CA at the PB/ZnO/COOH-MWNT electrode surface for successive addition of various concentrations of hydrogen peroxide in PBS of pH 7.0 at −0.004 V vs Ag/AgCl. The sensor achieved a steady state current within 4 sec after hydrogen peroxide spiking. Hydrogen peroxide concentration were detected as low as 1 μM. CA readings had a linear amperometric response with hydrogen peroxide in the 0.1 to 3.0 mM concentration region (FIG. 6B) with a limit of detection of 0.019±0.01 μM. The greatest deviation from linearity occurred at concentrations below 1 mM hydrogen peroxide (circled area in FIG. 6B). While not being bound by any particular theory or mechanism, it is postulated that the source of the deviation is due to residual Fenton-like reactions occurring at this concentration range at the electrocatalyst surface, decomposing the hydrogen peroxide to other ROS species. Lower concentrations of hydrogen peroxide appeared to be more susceptible to decomposition and less sensitive to it at higher hydrogen peroxide concentrations. In the present invention, the difficulties presented by this non-linear relationship are solved by incorporating the method of standard additions to chronoamperometry. Also, higher dynamic ranges for analysis may be achieved from this technique by incorporating serial dilutions in the SAM.

Validation control experiments showed that the calculated hydrogen peroxide concentrations precisely matched those of known spiked hydrogen peroxide solutions buffered to pH 7.0; FIG. 6C shows the overall results of the SAM CA for assaying hydrogen peroxide within 1-21 μM, an analysis range sufficient for probing hydrogen peroxide concentrations generated from oxidatively stressed cancer cells. Each data point in the plot corresponds to concentrations determined by a series of eight standard additions performed for each concentration point, repeated in triplicate to obtain the standard deviation error bars. Although there was deviation from linearity in the standard addition plots (correlation coefficients varied between R²=0.94-to-0.96), the overall results of the standard additions are in excellent agreement with the validation control (FIG. 6C).

As a benchmark for comparison, a recent study of MCF-10F, MCF-7 and MDA-MB-231 breast cancer cell lines that were oxidatively stressed using Dox-treatment showed hydrogen peroxide release within this concentration region (Pilco-Ferreto, N.; Calaf, M. Int. J Oncology 2016, 49, 753-762), which is comparable to the results for BT20 and 4T1 cell lines described herein. The Dox-treated BT20 and 4T1 cancer cells resulted in higher hydrogen peroxide concentration as compared to that of untreated cancer cells within the range of 6-to-14 μM.

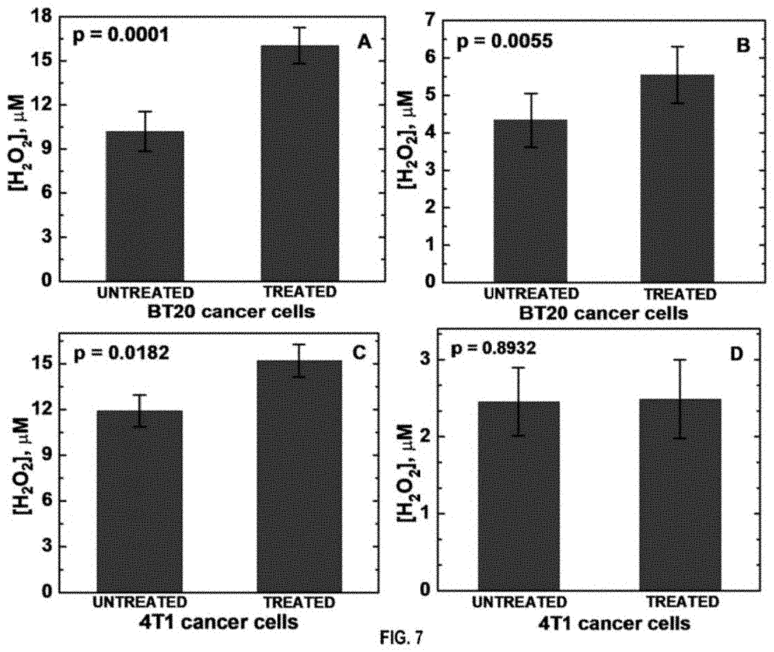

The analytical application of the PB/ZnO/COOH-MWNTs/GCE sensor towards hydrogen peroxide release from cells was performed using SAM CA. Standard additions were performed by spiking in known hydrogen peroxide concentrations and plotting the resulting current as a linear function of known concentration. Finally, the concentration of the analyte was determined using the x-intercept=−b/m) (Harris, D. C. Quantitative Chemical Analysis, 8th ed.; W.H. Freeman: New York, 2010; Westley, C.; Xu, Y.; Thilaganathan, B.; Carnell, J.; Turner, J.; Goodacre, R. Anal. Chem. 2017, 89, 2472-2477). This technique is particularly useful in cases in which the sample composition is complex in a changing matrix. The PB/ZnO/COOH-MWNT was applied to the GCE working electrode to determine hydrogen peroxide concentrations ranging from 1-to-21 μM in PBS before proceeding to the BT20 and 4T1 cancer cells. To validate the technique, the relationship between known and calculated concentrations of hydrogen peroxide was found with the correlation coefficient value, R²=0.9932 (FIG. 6C). FIG. 7 summarizes the assaying results before and after Dox treatment of BT20 and 4T1 cells using both CA SAM and ELISA.

BT20 and 4T1 cells were subject to oxidative stress to produce the hydrogen peroxide. CA SAM was carried out in untreated and 48 h Dox-treated BT20 cancer cells to compare the generation of hydrogen peroxide (FIG. 7A). There was higher hydrogen peroxide concentration in the Dox-treated samples (16.0±1.4 μM) (n=6) compared to the same untreated cancer cells (10.1 μM) (n=6) with the standard deviation of 1.2 μM in BT20 cancer cells (FIG. 7A). In comparison to ELISA assays (FIG. 7B), the same trend was observed.

Similarly, FIG. 7C also shows a higher concentration of hydrogen peroxide released (15.2 μM) (n=3) in Dox-treated 4T1 cancer cells (Table 2) as compared to the same untreated cancer cells (11.9 μM) (n=3). In comparing CA SAM measurements between Dox-treated and untreated 4T1 cells, measured hydrogen peroxide concentrations between these two samples are statistically significant (p=0.0182) (FIG. 7C). The corresponding assays between untreated and Dox-treated 4T1 cells with ELISA showed no ability to detect differences in hydrogen peroxide concentrations. ELISA results showed no statistical difference (p=0.8932) between Dox-treated and untreated 4T1 cells (FIG. 7D).

TABLE 2
Comparison of H2O2 (μM) from Dox
treated and untreated 4T1 cancer cells

| | |
|---|---|
| | ELISA results of |
| SAM CA for 4T1 cells | 4T1 cancer cells |

| Dox | Control | Dox | Control |
|---|---|---|---|
| treated | (untreated) | treated | (untreated) |
| 16 | 10.7 | 1.8289 | 1.9605 |
| 14 | 12.5 | 1.9605 | 2.0921 |
| 15.6 | 12.5 | 2.0921 | 2.2237 |
| | | 2.4868 | 2.2237 |
| | | 2.75 | 2.3553 |
| | | 2.8816 | 2.8816 |
| | | 2.6184 | 2.6184 |
| | | 3.0132 | 3.5395 |

CA SAM measurements were in excellent agreement in the measurement pattern of hydrogen peroxide concentration in both Dox-treated and untreated BT20 cancer cells with those of conventional ELISA method (Table S2) (n=8) as shown in FIG. 7B. Furthermore, the results indicate that the quantification of hydrogen peroxide under PB/ZnO/COOH-MWNTs is observed to be ˜2.5 times more sensitive than ELISA. Since hydrogen peroxide is a relatively unstable compound, its measurement needs to be performed quickly.

TABLE 3
Comparison of H2O2 (μM) from Dox-
treated and untreated BT20 cancer cells

| | |
|---|---|
| | ELISA results of |
| SAM CA for BT20 cells | BT20 cancer cells |

| Dox | Control | Dox | Control |
|---|---|---|---|
| treated | (untreated) | treated | (untreated) |
| 17.47 | 11.2 | 4.4091 | 4.258 |
| 15.1 | 12.2 | 5.0152 | 4.106 |
| 15.88 | 9.51 | 5.3182 | 3.652 |
| 17.47 | 10.59 | 5.3182 | 3.5 |
| 14.42 | 8.69 | 5.6212 | 4.409 |
| 15.88 | 8.99 | 5.6212 | 4.106 |
| | | 6.0758 | 4.864 |
| | | 6.9848 | 5.773 |

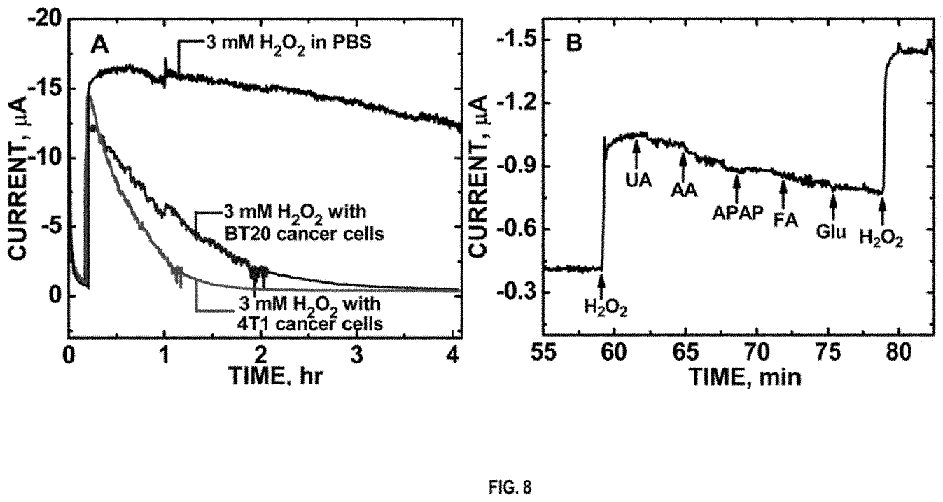

The CA SAM is not only significantly faster than ELISA, but also more sensitive for analyzing hydrogen peroxide as compared to ELISA. This improved assaying capability of CA SAM relative to ELISA is further corroborated by control measurements of 3 mM hydrogen peroxide within the BT20 and 4T1 cellular environment (FIG. 8A). A critical difference between the assaying techniques is analysis time. It should be noted that ELISA takes approximately 3 h to quantify the hydrogen peroxide in these cell media. In contrast, the CA SAM procedure for hydrogen peroxide the assay, employing the method of standard additions, takes 15-20 min to perform the aforementioned 8 standard additions for each concentration determination, which is a substantial decrease in analysis time during which hydrogen peroxide would decompose in the cancer cell media, hampering ROS mechanistic analysis. This substantial reduction in analysis time permits assaying before appreciable amounts of the hydrogen peroxide analyte decomposes. In addition, fluorescent compounds inherent to ELISA may contribute to hydrogen peroxide decomposition, resulting in lowered hydrogen peroxide readings. The CA SAM method is able to detect changes in hydrogen peroxide that are undetectable by ELISA in 4T1 cell line due to rapid decomposition of the hydrogen peroxide by these cell lines. Within the control CA experiment, current emanates from a 3 mM concentration of hydrogen peroxide in the presence of PBS solution, BT20, and 4T1 cell lines as a function of time (FIG. 8A), respectively. Hydrogen peroxide more rapidly decomposes in the presence of 4T1 and BT20 cancer cells than in PBS solution. The rate of decomposition is in descending order: 4T1>BT20>PBS. Although hydrogen peroxide decomposes more rapidly in the 4T1 cancer cell line as compared to BT20, CA SAM can still detect differences in production of hydrogen peroxide with the untreated and Dox-treated 4T1 cancer cells. In contrast, ELISA is not able to differentiate differences in hydrogen peroxide release between the Dox-treated and untreated 4T1 cells (FIG. 7D).

FIG. 8B shows CA responses the PB/ZnO/COOH-MWNT sensor against an array of interferents: uric acid (UA), ascorbic acid (AA), acetaminophen (APAP), folic acid (FA), and glucose (Glu). Experiments were carried out in PBS solution buffered to pH 7.0 at room temperature (25° C.). The CA responses were acquired at −0.004V (the reduction potential of hydrogen peroxide). Injection of 1.0 mM hydrogen peroxide into the reaction vessel resulted in a significant increase of current at the 58.5 min time point. Subsequent additions of 1.0 mM UA, 1.0 mM AA, 1.0 mM APAP, 1.0 mM FA, and 1.0 mM Glu at 3 min time intervals resulted in no observable signal increase. Finally, at the 79.5 min time point, 1.0 mM hydrogen peroxide was added into the electrochemical cell which showed no loss of signal upon addition of the interferents.

All references cited in this specification are herein incorporated by reference as though each reference was specifically and individually indicated to be incorporated by reference. The citation of any reference is for its disclosure prior to the filing date and should not be construed as an admission that the present disclosure is not entitled to antedate such reference by virtue of prior invention.

It will be understood that each of the elements described above, or two or more together may also find a useful application in other types of methods differing from the type described above. Without further analysis, the foregoing will so fully reveal the gist of the present disclosure that others can, by applying current knowledge, readily adapt it for various applications without omitting features that, from the standpoint of prior art, fairly constitute essential characteristics of the generic or specific aspects of this disclosure set forth in the appended claims. The foregoing embodiments are presented by way of example only; the scope of the present disclosure is to be limited only by the following claims.

1. A composition comprising an electrode having attached thereto a nanocomposite comprising a hydrogen peroxide catalyst and a zinc oxide nanostructure attached to a carboxylic acid-functionalized multiwalled carbon nanotube (ZnO/COOH-MWNT).

2. The composition of claim 1, wherein the electrode is a glass-like carbon electrode.

3. The composition of claim 1, wherein the hydrogen peroxide catalyst is Prussian blue.

4. The composition of claim 1, wherein the zinc oxide nanostructure has an average diameter of about 50 nm to about 60 nm.

5. The composition of claim 1, wherein the carboxylic acid-functionalized multiwalled carbon nanotube has a diameter of about 30 nm.

6. The composition of claim 3, wherein the ratio of Prussian blue to ZnO/COOH-MWNT is about 1:2.

7. A method of preparing a nanocomposite, comprising:
d) preparing a zinc oxide nanostructure;
e) attaching the zinc oxide nanostructure to a carboxylic acid-functionalized multiwalled carbon nanotube; and
f) attaching a hydrogen peroxide catalyst to the carboxylic acid-functionalized multiwalled carbon nanotube.

8. The method of claim 7, wherein (b) is performed by ultrasonication for approximately 60 minutes.

9. The method of claim 7, wherein (c) is performed at a pH of 6.6.

10. The method of claim 9, wherein (c) is performed over a period of about 5 hours.

11. The method of claim 7, wherein the hydrogen peroxide catalyst is attached to the nanotube electrostatically.

12. The method of claim 7, wherein the hydrogen peroxide catalyst is Prussian blue.

13. The method of claim 7 further comprising depositing the nanocomposite onto an electrode.

14. The method of claim 13, wherein the electrode is a glass-like carbon electrode.

15. A method for quantitating the level of hydrogen peroxide in a biological sample, the method comprising:
c) generating a standard curve for hydrogen peroxide concentration by
i. adding serial concentrations of hydrogen peroxide to a buffer solution,
ii. inserting the electrode of claim 1 into the solution,
iii. measuring the concentration of hydrogen peroxide using an electrochemical sensor, and
iv. plotting the resulting current at each concentration of hydrogen peroxide to generate the standard curve; and
d) determining the concentration of hydrogen peroxide in the biological sample by
i. inserting the electrode of claim 1 into the biological sample,
ii. detecting hydrogen peroxide through the electrode using an electrochemical sensor, and
iii. determining the concentration of hydrogen peroxide by comparing the results of (b)(ii) to the standard curve.

16. The method of claim 15, wherein the electrochemical sensor is a cyclic voltammeter or a chronoamperometer,

17. The method of claim 15, wherein hydrogen peroxide is detected in the biological sample at a concentration of at least 1 μM.

18. The method of claim 15, wherein the biological sample is from a subject having or suspected of having cancer, or is an immortalized cancer cell line.

19. The method of claim 18, wherein the cancer is breast cancer.

20. The method of claim 15, wherein the hydrogen peroxide is detected in the biological sample in the range of about 1 μM to about 21 μM.

21. The method of claim 15, wherein the hydrogen peroxide is quantitated in the biological sample within about 15 minutes.

22. The method of claim 15, wherein no matrix effect is observed.
